(19) United States
(12) Patent Application Publication
(10) Pub. No.: US 2023/0357132 A1
(43) Pub. Date: Nov. 9, 2023

(54) SYNTHESIS OF mRNA DELIVERY AGENT

(71) Applicant: WISDOM PHARMACEUTICAL CO., LTD., Nantong (CN)

(72) Inventors: Ping Zou, Nantong (CN); Zhiwei Zuo, Nantong (CN); Xiaolong Qiu, Nantong (CN); Tao Xu, Nantong (CN); Lin Hu, Nantong (CN); Lingling Chu, Nantong (CN); Wenbo Liu, Nantong (CN)

(73) Assignee: WISDOM PHARMACEUTICAL CO., LTD., Nantong (CN)

(21) Appl. No.: 17/630,569

(22) PCT Filed: Nov. 19, 2021
(86) PCT No.: PCT/CN2021/131832

§ 371 Date: Jan. 27, 2022

(30) Foreign Application Priority Data
Jul. 16, 2021 (CN) 202110804660.5

(51) Int. Cl. C07C 229/16; A61K 48/00

(52) U.S. Cl. CPC C07C 229/16; A61K 48/0033

(57) ABSTRACT

The present disclosure relates to synthesis of an mRNA delivery agent, in particular to a reaction of a related amino compound with ethylene oxide in the presence of ytterbium triflate.

### CROSS REFERENCE TO RELATED APPLICATION(S)

This patent application claims the benefit and priority of Chinese Patent Application No. 202110804660.5 filed on Jul. 16, 2021 and entitled “SYNTHESIS OF mRNA DELIVERY AGENT”, the disclosure of which is incorporated by reference herein in its entirety as part of the present application.

### TECHNICAL FIELD

The present disclosure relates to the technical field of chemical synthesis, in particular to synthesis of an mRNA delivery agent.

### BACKGROUND ART

The pandemic of COVID-19 pushes mRNA vaccines into the central stage of biotechnology and pharmaceutical industry. The speed of vaccine development has exceeded expectations, and vaccines have become available 10 months after publishing the SARS-CoV-2 sequence. This success not only demonstrates that biotechnology and pharmaceutical industry can cope with urgent and unsatisfied global demands, but also indicates the inherent capability of mRNA serving as a pharmaceutical form. Compared with conventional inactivated vaccines, mRNA vaccines have advantages of low cost, high production efficiency, and high safety, and possess a potential to synthesize any protein. Therefore, mRNA vaccines have enormous application potential to fight against novel infectious viruses that conventional vaccines could no longer cope with. However, use of mRNA vaccines is always limited due to the instability, high innate immunogenicity, and low in vivo delivery efficiency of mRNA molecules. To achieve the wide use of mRNA vaccines, it is necessary to focus on the delivery technology. mRNA vaccines need suitable delivery carriers (delivery agent, also known as transfer agent) to deliver them into the body in order to obtain a better immune effect. Therefore, developing an efficient and nontoxic delivery system is the key to success of mRNA vaccines. Prof. Michael D. Buschmann, head of the Bioengineering Department of the George Mason University, elaborated the development of mRNA delivery systems, summarized results of preclinical and clinical studies of SARS-CoV-2 mRNA vaccines, emphatically introduced lipid nanoparticles used in current clinical trials of SARS-CoV-2 vaccines, and analyzed the use of lipid nanoparticles in mRNA vaccines.

Before COVID-19, mRNA vaccines have been used in preclinical and clinical studies on, for example, influenza, Zika virus, HIV, Ebola virus, rabies, malaria, genital herpes, and toxoplasmosis. In the current competition of COVID-19 vaccines, based on the initial success of mRNA vaccines, there are eight on-going human trials of mRNA vaccines, which are led by BioNTech/Pfizer, Moderna, CureVac, Sanofi/TranslateBio, Arcturus/Duke NUS, Imperial College London, Chula-longkom University, and Providence Therapeutics. It is worth noting that two of these trials have published midterm results of phase III trials, reporting the mRNA sequence encoding the spike protein immunogen (delivered in the form of lipid nanoparticle) and the efficacy of a >94% decrease in SARS-CoV-2 infection rate after two doses of 30 or 100 μg.

Delivery technology platform is one of the keys to mRNA vaccines, and a number of mRNA preparation systems have been reported, and most of which have been in clinical trials. These preparation technologies realize the delivery of mRNA vaccines by forming special mRNA carriers. These vector technologies include: protamine carrier technology, lipid nanoparticle carrier technology, and polymeric carrier technology.

At present, the lipid nanoparticle carrier technology is the most widely used in the current development and production of SARS-CoV-2 vaccines. Delivery agents used in the lipid nanoparticle carrier technology generally include compounds having the structure of the following formula I, their salts, or their isomers.

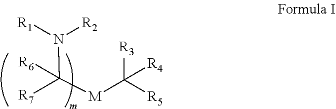

Chinese Patent Application No. CN109476718 further describes a general molecular formula of these lipid nanoparticle delivery agents, and the specific general molecular formula is shown in formula II below.

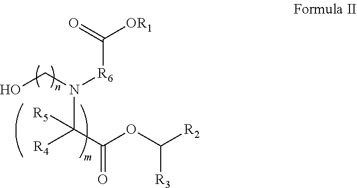

Some structures of representative delivery agents are mentioned in the description and examples in Chinese Patent Application No. CN109476718, such as some compounds having the following structures:

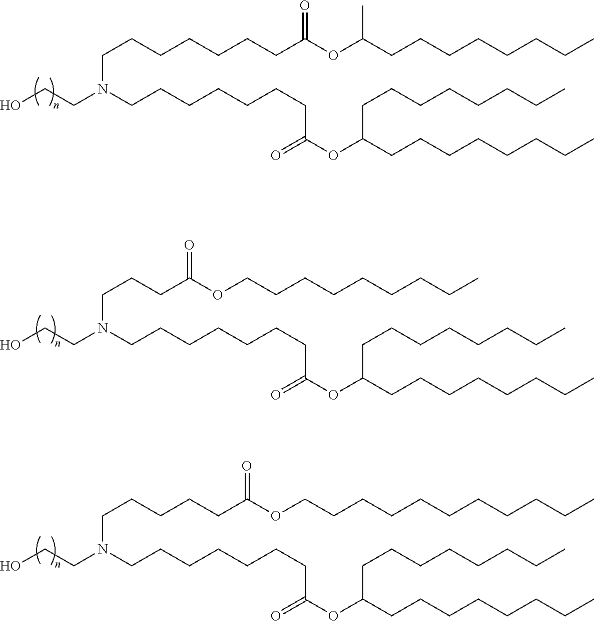

For the synthesis of some delivery agents with n =1, the strategy adopted by the patent is: conducting a condensation reaction of a compound of formula III with a compound of formula IV to prepare a compound of formula V; conducting a nucleophilic substitution reaction of the compound of formula V with aminoethanol, where bromine in the compound of formula V is nucleophilically substituted by amino group in the aminoethanol to obtain a compound of formula VI; and finally conducting a nucleophilic substitution reaction of the compound of formula VI with a compound of formula VII to prepare a compound of formula VIII. This synthetic route can achieve the synthesis of the delivery agent, but the exposed amino group contained in the product compound of formula VI from the second step of the reaction can continue to undergo a nucleophilic substitution reaction with the compound of formula V to produce dimeric impurities in the reaction mixture; meanwhile, in the third step of the reaction, the final product compound of formula VIII continue to react with the compound of formula VII to produce quaternary ammonium bromide impurities very easily. Defects of these reaction routes lead to a bottleneck of this synthetic route in the process of industrialization.

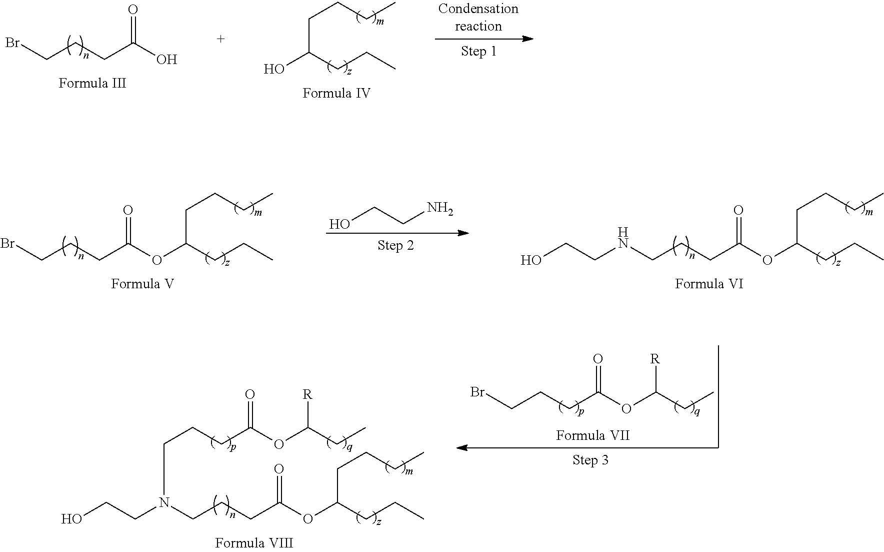

### SUMMARY

An objective of the present disclosure is to provide a new method for preparing an mRNA delivery agent, in order to synthesize a compound of formula XI.

A synthetic route of this method is a reaction of an amino compound of formula X with ethylene oxide in the presence of a solvent and an additive to facilitate the preparation of the compound of formula XI.

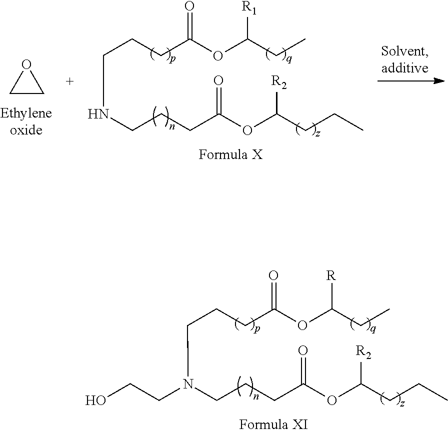

The solvent used in the reaction may be selected from the group consisting of acetonitrile, dioxane, tetrahydrofuran (THF), dimethyl formamide (DMF), dimethylsulfoxide (DMSO), and 2-methyltetrahydrofuran (2-Me-THF).

The additive used in the reaction may be ytterbium trifluoromethanesulfonate hydrate.

R₁ in the formulas X and XI may be selected from the group consisting of H and C₁-C₁₀ alkyl, and R₂ in the formulas X and XI may be selected from the group consisting of H and C₁-C₁₀ alkyl.

In the formulas X and XI, n may be 2-10, z may be 1-10, p may be 2-10, and q may be 1-11.

### DETAILED DESCRIPTION OF THE EMBODIMENTS

The present disclosure will be described with the following typical example. All simple substitutions and improvements of the present disclosure made by those skilled in the art are included in the technical solutions claimed by the present disclosure.

### Example 1: Preparation of 8,8′-((2-hydroxyethyl)azanediyl)dinonyl dicaprylate.

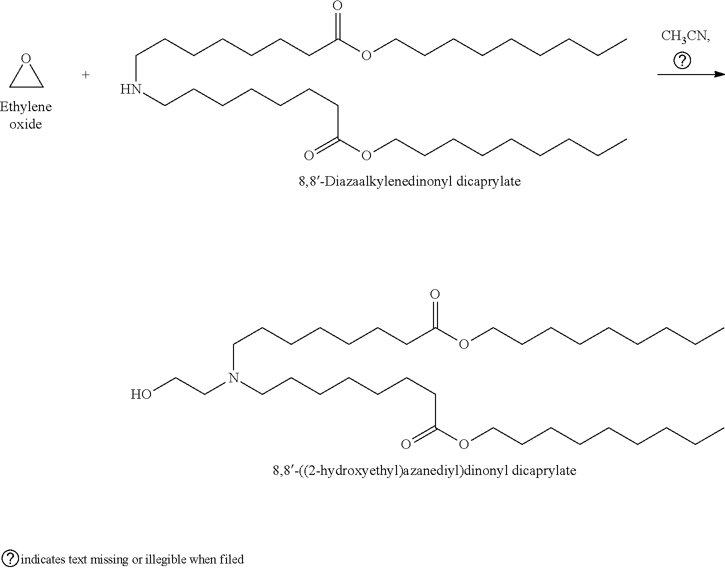

8,8′-Diazaalkylenedinonyl dicaprylate (10.0 g, 18.05 mmol) and acetonitrile (50 mL) were added successively to a four-neck round-bottom flask. After addition, the system was cooled down to −80° C., and the reaction system was bubbled with ethylene oxide (20 g, 0.45 mol); subsequently, ytterbium triflate (1.12 g, 1.81 mmol) was added to the reaction system. After addition, the reaction system was naturally heated to room temperature for reaction under stirring, and the reaction was tracked by thin layer chromatography (TLC) till the complete consumption of the starting material 8,8′-diazaalkylenedinonyl dicaprylate. After the reaction, water (100 mL) was added to the system to quench the reaction; the reaction system was extracted with ethyl acetate (3×80 mL); the organic phase was combined, dried over anhydrous sodium sulfate, and filtered; the solvent was removed from a filtrate under reduced pressure, and residues were purified by column chromatography (CH₂Cl₂/MeOH=30:1) to obtain 8,8′-((2-hydroxyethyl)azanediyl)dinonyl dicaprylate (9.18 g, 85.1%). ¹H NMR (600 MHz, CDCl₃) δ0.90 (m, 6H), 1.02-1.75 (m, 49H), 2.31 (m, 4H), 2.72-2.41 (m, 6H), 3.61 (m, 2H), 4.07 (m, 4H) Mass: 599 [M+H]⁺.

Although the present disclosure is described in detail in conjunction with the foregoing example, it is only a part of, not all of, the examples of the present disclosure. Other examples can be obtained by persons based on the example without creative efforts, and all of these examples shall fall within the protection scope of the present disclosure.

1. A method for preparing an mRNA delivery agent, which is specifically a compound of formula XI, the method comprises reacting an amino compound of formula X with ethylene oxide in the presence of a solvent and an additive, and a reaction formula is as follows: 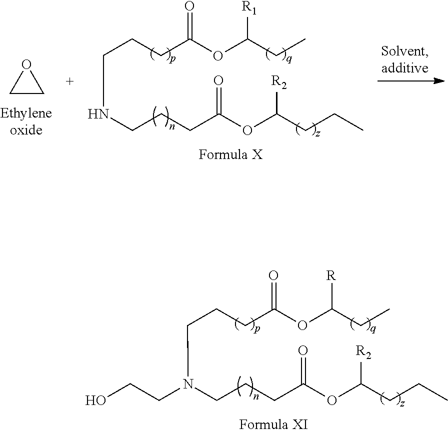
wherein
R₁ in the formulas X and XI is selected from the group consisting of H and C₁-C₁₀ alkyl, and R₂ in the formulas X and XI is selected from the group consisting of H and C₁-C₁₀ alky,
in the formulas X and XI, n is in the range of 2-10, z is in the range of 1-10, p is in the range of 2-10, and q is in the range of 1-11.

2. The method according to claim 1, wherein the solvent is selected from the group consisting of acetonitrile, dioxane, tetrahydrofuran (THF), dimethyl formamide (DMF), dimethylsulfoxide (DMSO), and 2-methyltetrahydrofuran (2-Me-THF).

3. The method according to claim 1, wherein the additive is ytterbium triflate.

4. (canceled)

5. (canceled)

6. The method according to claim 1, wherein the reaction is followed by the following steps: adding water to a system to quench the reaction, extracting the reaction system with ethyl acetate, combining an organic phase, drying the organic phase over anhydrous sodium sulfate, filtering, removing the solvent from a filtrate under reduced pressure, and purifying residues by column chromatography, to obtain the compound of formula XI.

7. The method according to claim 6, wherein an eluent for the column chromatography is dichloromethane and methanol at a volume ratio of 30:1.

8. An mRNA delivery agent wherein the agent is a compound having a structural formula shown in formula XI 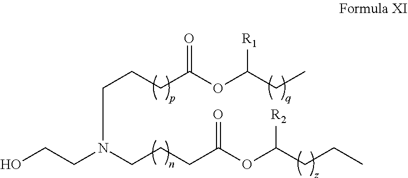
wherein
R₁ is selected from the group consisting of H and C₁-C₁₀ alkyl, and R₂ is selected from the group consisting of H and C₁-C₁₀ alky;
n is in the range of 2-10, z is in the range of 1-10, p is in the range of 2-10, and q is in the range of 1-11.

9. The mRNA delivery agent compound according to claim 8, wherein the compound is 8,8′-((2-hydroxyethyl)azanediyl)dinonyl dicaprylate.

10. (canceled)
